(19) United States
(12) Patent Application Publication
(10) Pub. No.: US 2007/0117090 A1
(43) Pub. Date: May 24, 2007

(54) Processing nucleic acid

(76) Inventor: Henry Charlton, Salisbury (GB)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 10/495,892

(22) PCT Filed: Nov. 19, 2002
(86) PCT No.: PCT/GB02/05215

§ 371 Date: Nov. 16, 2004

(30) Foreign Application Priority Data
Nov. 20, 2001 (GB) 0127803.5

(51) Int. Cl. C12Q 1/68; C12N 1/08

(52) U.S. Cl. 435/6; 536/25.4; 435/270

(57) ABSTRACT

The present invention relates to a method of processing nucleic acid. More particularly, it relates to a method of purifying extra-chromosomal DNA by removing cell debris and/or RNA from a process stream comprising extra chromosomal DNA and a precipitate resulting from preceding cell lysis and/or precipitation reactions. It also relates to nucleic acid, particularly extra chromosomal DNA, purified by a method of the invention; a pharmaceutical composition comprising or consisting of the same and apparatus for said method. The method comprises: Controlling the cell lysis and/or precipitation reactions to substantially minimise the formation of small particles and/or maximise the formation of large particles; and Straining the process stream by passing it through a mesh or sieve with a mesh size of greater than 75 μm to remove a substantial % mass of the precipitate from the process stream.

### FIELD OF THE INVENTION

The present invention relates to a method of processing nucleic acid. More particularly, it relates to a method of purifying extra-chromosomal DNA by removing cell debris and/or RNA from a process stream comprising extra chromosomal DNA and a precipitate. It also relates to nucleic acid, particularly extra chromosomal DNA, purified by a method of the invention; a pharmaceutical composition comprising or consisting of the same and apparatus for said method.

### BACKGROUND TO THE INVENTION

Large scale manufacturing of plasmid DNA is being developed to enter new areas of healthcare whereby integration of genetic material into host cells can elicit a therapeutic effect.

In the manufacture of plasmid DNA, the plasmid DNA is obtained from cells by lysing them. The plasmid DNA is then separated from the cell debris and purified. The first step of this purification process often comprises the steps of precipitating cell debris and filtering the process stream to separate the solids.

The large quantities of contaminant are typically precipitated from the process stream by the addition of potassium acetate to chemically lysed bacterial cells.

In the large-scale manufacture of plasmid DNA, one of the steps involves removal of a precipitate from the liquid fraction of the process stream. This precipitate is formed by the mixing of chilled, typically, 3M potassium acetate with typically, E.coli cells that have been previously lysed by mixing with 0.96 M NaOH and 6% (w/v) sodium dodecyl sulphate (SDS). This precipitate is shear-sensitive, i.e. it will readily fragment into smaller particles when exposed to mechanical work such as passage through pumps or pipes above a certain speed or rate. At this stage of manufacture, the solids content of the process stream can be up to 20% (w/v) which in comparison to other bio-processes is a large amount of solids to remove. The precipitate may sink or float, depending on the duration and intensity of the mixing step during which potassium acetate is added, and the presence of other salts. Nevertheless, the particles must be removed in order to allow further purification of the plasmid. Solids removal in bio-processes normally takes place by either centrifugation e.g. batch centrifugation, filtration, or, more unusually, by settling.

The centrifugation step succeeds in removing precipitate, but has limitations. First, the batch centrifuge can only handle 6 litres per 20-minute run, so the maximum throughput for any machine is no greater than 18 litres per hour. Second, the operation of these machines is labour intensive. Hence, this part of the process is not readily scalable: a 1000-litre batch would require 56 centrifuges (each one costing approximately £16,000) and a correspondingly large workforce to process the volume in the same time. Third, the process is not integrated and there are periods when process fluids are exposed to the air, which requires increased management of air flow and cleanliness in the appropriate parts of the manufacturing facility. Fourth, the centrifugation step does not always remove all the particles, and the process fluid must pass through a filtration train in order to guarantee the clarity of the process fluid and protect subsequent downstream operations.

It is an aim of the present invention to overcome these problems by designing a scalable, rapid, low-shear, inexpensive method by which to remove precipitate from a process stream containing plasmid DNA.

The applicant has determined that the size of the precipitate is dependent on the preceding processing steps such as, for example, the duration and rate of agitation of the cell lysis, and/or precipitation reactions, and that furthermore, the size of the particles can have a significant effect on downstream processing steps. By controlling the precipitate size, they are able to optimise its economic removal in the subsequent downstream process, and by using a mesh or sieve (rather than a traditional filter device, such as, for example a bag filter, membrane or fabric) they are able to remove the precipitate very effectively. More specifically, it enables them to remove large quantities of precipitate quickly and simply. It also reduces the mechanical work done on the precipitate. The system can also be configured such that precipitate will be removed, irrespective of whether it sinks or floats in the liquor.

### SUMMARY OF THE INVENTION

According to a first aspect of the present invention, there is provided a method of purifying extra-chromosomal DNA, by removing cell debris and/or RNA precipitate from a process stream comprising extra chromosomal DNA and cell debris precipitate, and/or RNA precipitate, comprising:
a. Controlling the cell lysis and/or precipitation reactions to substantially minimise the formation of small particles, and/or maximise the formation of large particles; and
b. Straining the process stream by passing it through a mesh or sieve with a mesh size of greater than 75 μm, to remove a substantial % mass of the precipitate from the process stream.

Preferably, the method comprises both minimising the formation of small particles and maximising the formation of large particles.

The terms small and large particles are relative terms, and are used in context to describe the effect of relative particle size on the ability to purify a process stream.

More particularly, small particles are those retained by a mesh size of 53 μm, but which pass through a mesh size of 150 μm. Whilst some particles will pass through the mesh size of 53 μm, these are not included in the calculations or considered to be small particles in the context of this application. Thus, the calculations assume 100% by weight of the particles are those captured by a mesh or sieve with a mesh size of 53 μm.

Preferably the small particles comprise no more than 15% by weight of the total weight, more preferably still, less than 13% and most preferably, less than 11%.

More particularly, the large particles are those retained by a mesh size of 425 μm.

Preferably, the process stream comprises at least 60% by weight of large particles; more preferably still, at least 65%; more preferably still, at least 70% and most preferably still, at least 75%.

Preferably, the process stream further comprises intermediate-sized particles. The intermediate-sized particles are those retained by a mesh size of 150 μm, but which pass through a mesh or sieve with a mesh size of 425 μm.

Preferably, the process stream comprises less than 20% by weight of the intermediate particles; more preferably still, less than 18% and most preferably, less than 16%.

More preferably, the process stream comprises at least 50% by weight of the particles that are retained by a mesh or sieve with a mesh size of 850 μm; more preferably still at least 60%, and most preferably at least 65%.

Preferably, the process stream is passed through a mesh or sieve with a mesh size of greater than 75 μm; more preferably still, greater than 150 μm and most preferably about 200 μm or greater.

In one embodiment, the process stream is passed through a single sieve and in another embodiment, the process stream is passed through a series of sieves of decreasing mesh size.

Preferably, the mesh or sieve comprises a contact face that lies substantially wholly planar to the process stream.

Preferably, the contact face is substantially rigid. In other words it is not flexible like a bag filter.

Preferably the sieve or mesh is made of metal, most preferably stainless steel. This facilitates easy cleaning.

One way of controlling the size of the particles is to control the rate of agitation in the cell lysis, and/or precipitation reactions, to minimise the formation of small particles and maximise the formation of large particles. The preferred rate of agitation is achieved by maintaining a tip speed of less than 1.15 m/s; more preferably, less than 1 m/s; more preferably still, less than 0.5 m/s and optimally abut 0.30 m/s.

The tip speed is linked to rpm by the relationship tip speed equals circumference of agitator multiplied by rate of revolution (rpm). Thus, a larger agitator operating at the same rpm as a smaller agitator has a faster tip speed.

In addition to the rate of agitation, it is preferred to control the duration of agitation. It is preferred to minimise the duration of agitation during the cell lysis, and/or precipitation reactions, to minimise the formation of small particles and maximise the formation of large particles. Preferably, the period is less than 1 hour; more preferably still, less than 30 minutes; most preferably a few minutes. Indeed, it may be for as little as a few seconds.

Other methods that reduce shear, such as static or vortex mixing can also be used.

The process may comprise a number of further purification steps, such as, for example, passing the process stream through a depth filter; such as, for example, a 0.2 μm filter membrane.

An advantage of the method described is that it omits a centrifugation step to remove particles derived from the lysis and precipitation reactions.

The preferred extra chromosomal DNA is plasmid DNA.

The process is particularly suited to a large-scale process, typically one handling at least 10 litres of starting liquid. More preferably, the process handles 50 litres or more.

The process may be gravity-fed, or it may be operated under the application of pressure. It is preferred to use gravity feed, since this reduces shear.

The design of the agitator can also affect the process, and it is preferred to use an agitator comprising an impellor with large blades. The design of blade can affect the plasmid DNA, and an impellor comprising a plurality of blades, preferably four arranged as two pairs of diametrically opposed blades, each inclined and overlapping one another. Preferably, the blades are pitched at an angle of from 40-80 degrees to the horizontal; more preferably still, from 50 to 70 degrees, and most preferably, about 60 degrees. The blades should extend in length to fill at least 40% the height of the vessel.

The blades should not be fully covered during the lysis step.

According to a further aspect, the invention extends to extra chromosomal DNA, purified by the method of the invention and a pharmaceutical composition comprising or consisting of extra chromosomal DNA purified by the method.

Other aspects include a filter plate comprising a mesh or sieve of greater than 75 μm for use in the process of the invention, and an agitator comprising an impellor for a large-scale vessel comprising blades which extend in length to fill at least 40% of the vessel.

The mesh or sieve preferably comprises a filter mesh and a support mesh. The filter mesh preferably has a mesh size of greater than 75 μm; more preferably, greater than 150 μm, and most preferably, about or greater than 200 μm. Preferably, it is made of stainless steel and is most preferably a wire mesh disc. To provide a degree of rigidity and to help to hold it flat, the filter mesh is preferably supported on a support mesh. The support mesh is preferably made of stainless steel, and may be, for example, a 1.6 mm aperture wire mesh disc. In order to help overcome a problem of locating the filter mesh and support mesh on a filter plate, the filter mesh and support mesh are preferably welded, or otherwise held together as one. The mesh or sieve is preferably provided with a seal on its underside to assist with location and prevent (in use) bypass of liquid. This seal is preferably in the form of an O-ring. Preferably, the mesh or sieve has a diameter of greater than 0.50 m, and is preferably about 1.0 m in diameter, and is circular in plan view.

In use, the mesh or sieve is supported in a filter holder adapted for use in a GMP environment. The filter holder may, for example, be in the form of a trolley-mounted plate filter vessel.

The mesh or sieve and the design of a filter holder are described further in the specific examples.

A sieve arrangement, as disclosed herein, is particularly advantageous in purifying plasmid DNA, in that it can be used in a single step clarification/purification process in which significant amounts of cell debris and RNA are removed along with, to a lesser degree, other impurities such as protein, endotoxins, and genomic DNA.

To explain: in purifying plasmid DNA, it is standard practice to clarify a lysed material by centrifugation to remove cell debris. A typical process for extracting & purifying plasmid DNA is that of Birnboim and Dolby (1979). This method makes use of the observation that there is a narrow range of pH (12.0 to 12.5) within which denaturation of linear DNA, but not covalently closed circular DNA, occurs. Plasmid containing cells are often treated with lysozyme to weaken the cell walls, and then lysed with SDS. Chromosomal DNA remains in a high molecular weight form, but is denatured. Upon neutralisation with acidic sodium or potassium acetate, the chromosomal DNA renatures and aggregates to form an insoluable network. Simultaneously, the high concentration of the acetate causes precipitation of protein-SDS complexes and high molecule weight RNA. Provided the pH of the alkaline denaturing step is carefully controlled, the ccc plasmid DNA remains in solution whilst much of the contaminating macro molecules are precipitated.

This precipitate can be retained using the sieve arrangement disclosed as opposed to using centrifugation.

Whereas typically, a clarified lysate (obtained by centrifugation) may then undergo further purification steps, e.g calcium chloride precipitation, to remove high levels of RNA. The applicant has determined that a calcium chloride precipitation step can be run in conjunction with the traditional clarification of lysed cells and a single precipitate collection step undertaken which precipitate contains cell debris and high molecular weight RNA.

According to yet a further aspect of the present invention, there is provided a method of purifying extrachromosomal DNA from a process stream comprising lysed cells said method comprising:
i) neutralising the process stream comprising lysed cells with, for example, sodium or potassium acetate;
ii) adding a high concentration of an antichaotropic salt, for example calcium chloride, to precipitate out RNA; and
iii) separating the solids from the process stream by passing the process stream through a mesh or sieve with a mesh size greater than 75 μM.

The various aspects of the invention will now be described, by way of example only, with reference to the following examples, tables and figures.

### BRIEF DESCRIPTION OF THE DRAWINGS

FIG. 1 is a photograph of precipitated lysate taken from a 1 litre lab-scale reaction, stirrer speed was 100 RPM for both the lysis and precipitation reactions;

FIG. 2 is a photograph of precipitated lysate taken from a 50 litre full-scale reaction;

FIG. 3 is a graph showing the mass of precipitate held on different sized sieves used to fractionate particles on the basis of size;

FIG. 4 is a table showing the data obtained under different agitation conditions (impeller tip speed and duration) and includes the data by mass, percentage mass and as subsets by sieve size;

FIG. 5 is a plot of the data showing mass distribution against sieve size for 850 μm; 425 μm; 212 μm; 150 μm; 75 μm; and 53 μm;

FIG. 6 is a plot of the data showing % mass against sieve size for 850 μm; 425 μm; 212 μm; 150 μm; 75 μm; and 53 μm;

FIG. 7 is a plot of the data showing % mass against sieve size for 850 μm+425 μm; 212 μm+150 μm; and 75 μm+53 μm;

FIG. 8 is a plot of the data showing % mass against sieve size for 850 μm+425 μm+212 μm and +150 μm+751 μm+531 μm;

FIG. 9 is a plot of the data showing % mass against sieve size for 850 μm+425 μm+212 μm+150 μm; and 75 μm+53 μm;

FIG. 10 is a plot of the data showing % mass against sieve size for 850 μm+425 μm and 212 μm+150 μm+75 μm+53 μm;

FIG. 11 is a plot showing plasmid yield as a function of lysis time (various agitation rates);

FIG. 12 is a plot showing plasmid yield as a function of precipitation time (various agitation rates);

FIG. 13 is a plot showing particle size of material from large and small-scale experiments;

FIG. 14 is a side view of a filtration unit according to one aspect of the invention;

FIG. 15 is a view from above of the unit of FIG. 14;

FIG. 16 is a part cross sectional view of a filter plate, cover assembly and mesh or sieve of the filtration unit; and

FIG. 17 illustrates a perspective view from one side and above of an agitator assembly according to a further aspect of the invention.

### DETAILED DESCRIPTION OF THE INVENTION

Nature of the Precipitate: Full-Scale and Lab Scale Differences

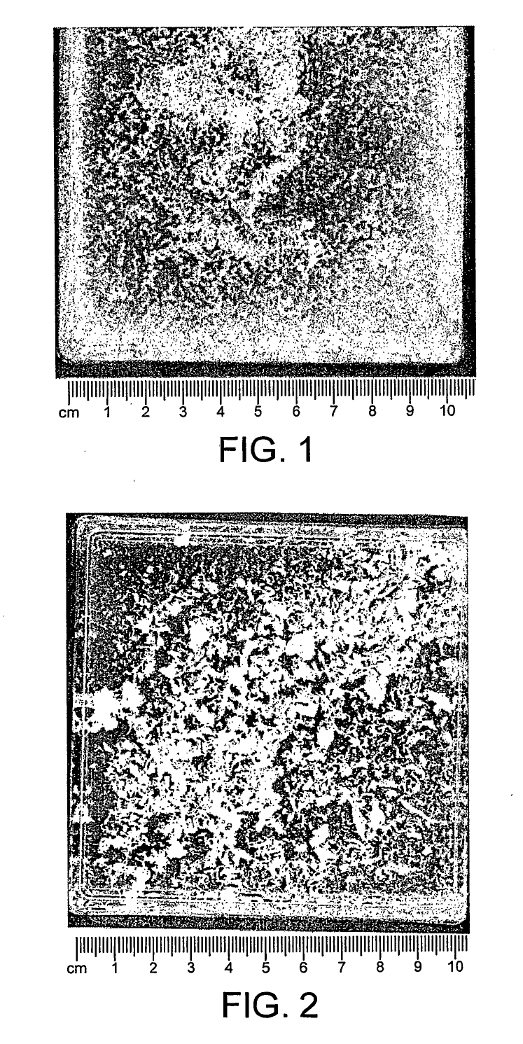

The initial studies to aid in the design of the integrated precipitate removal step were based on assessing the size of precipitate that was typically produced in the lysis/precipitation reaction. Samples were taken from both lab scale (1 Litre) and process scale (10 or 50 Litre) equipment, and it was readily apparent that there was a significant difference in the size of particles generated at the different locations. The results are illustrated in FIGS. 1 and 2. FIG. 1 is a photograph of precipitate taken from a 1 litre lab-scale reaction, stirrer speed 100 rpm for both lysis and precipitation reactions (tip speed 0.3 m/s). FIG. 2 is a photograph of precipitate taken from a 50 litre large-scale reaction, stirrer speed 50 rpm for lysis and 80 rpm for precipitation.

Clearly, the material from the full-scale reaction is smaller than that from the lab-scale reaction, and it was decided to identify the parameters that govern the formation of precipitate of a specific size. However, there was no readily available method by which to quantify differences in the size of the precipitate (rather than relying on visual inspection), so a method was developed by which to do this.

Quantification of Precipitate Size Differences

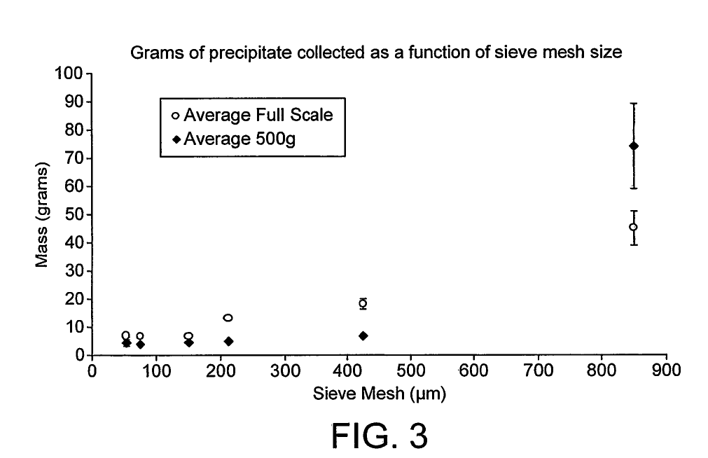

One method of grading material with size differences is by using a stack of sieves. Traditionally, this has been applied in industries that process dust and rock etc. The mesh size of each sieve sequentially decreases with the height of the stack. By applying a sample to the top of a stack of sieves of mesh size 850 μm, 425 μm, 212 μm, 150 μm, 75 μm and 53 μm particles, with a size greater than the mesh size but lower than the sieve above, are held in that layer. By weighing each sieve after settling, the material can be graded for its particle size distribution by mass. This method of particle sizing was applied to the precipitate taken from small scale experiments and the full-scale runs, and the data is given in FIG. 3. The difference between the two samples can be demonstrated quantitatively, as well as visually.

Factors Impacting on Precipitate Size

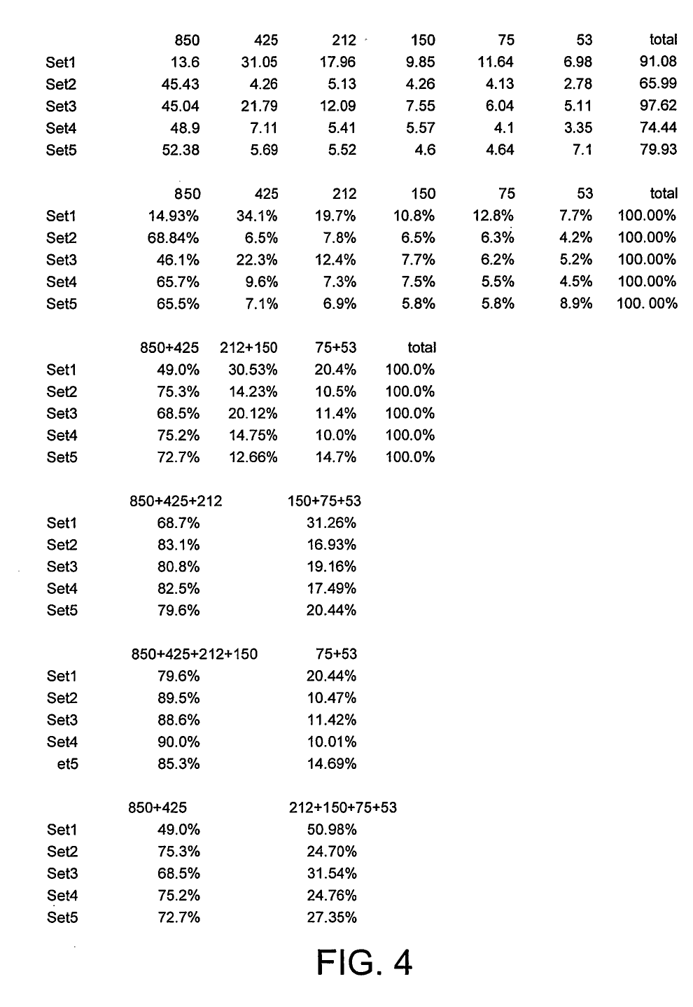

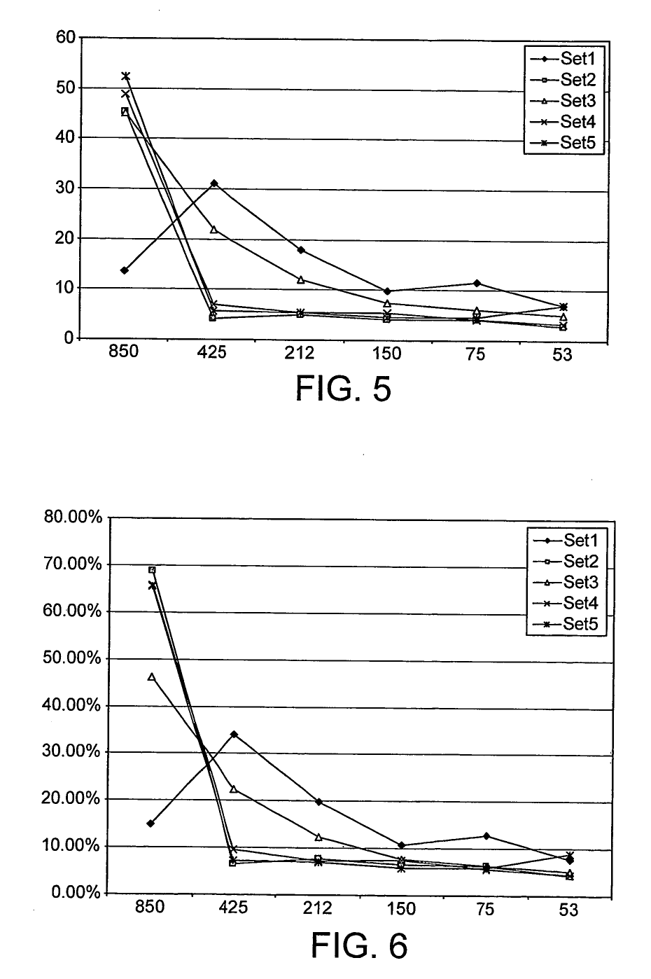

Given the evident difference in particle size distribution between the laboratory scale and full-scale equipment, it was decided to perform a series of experiments to try and determine those factors governing the difference in observed results. One difference between the two systems, other than scale, was the tip speed of the agitator employed to mix the contents of the lysis vessel. The maximum tip speed of the agitator in the large-scale vessel at 80 RPM was 1.15 m s⁻¹ and to match this tip speed, this equated to 220 RPM in the 1 litre lab-scale runs. Generally, 100 RPM had been used to perform the 1 litre scale experiments. Therefore, a range of experiments with various tip speeds and for various durations, was conducted. The resultant data is shown in FIG. 4 (Table 1). The data was manipulated to give % distribution figures (by weight) and the data plotted for the different runs based on the size distribution (FIGS. 5 to 10).

FIG. 5 compares the weight distribution across all mesh sizes.

FIG. 6 compares the % weight distribution across all mesh sizes.

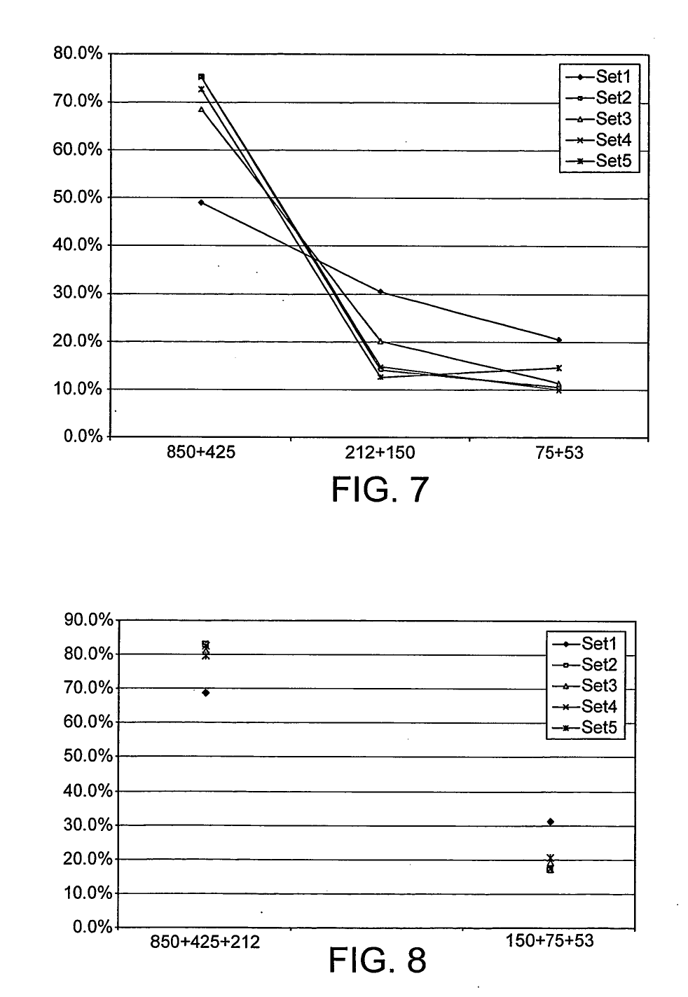

FIG. 7 groups the results into large, intermediate and small particle size.

FIG. 8 compares the results by separation using a 212 μm mesh.

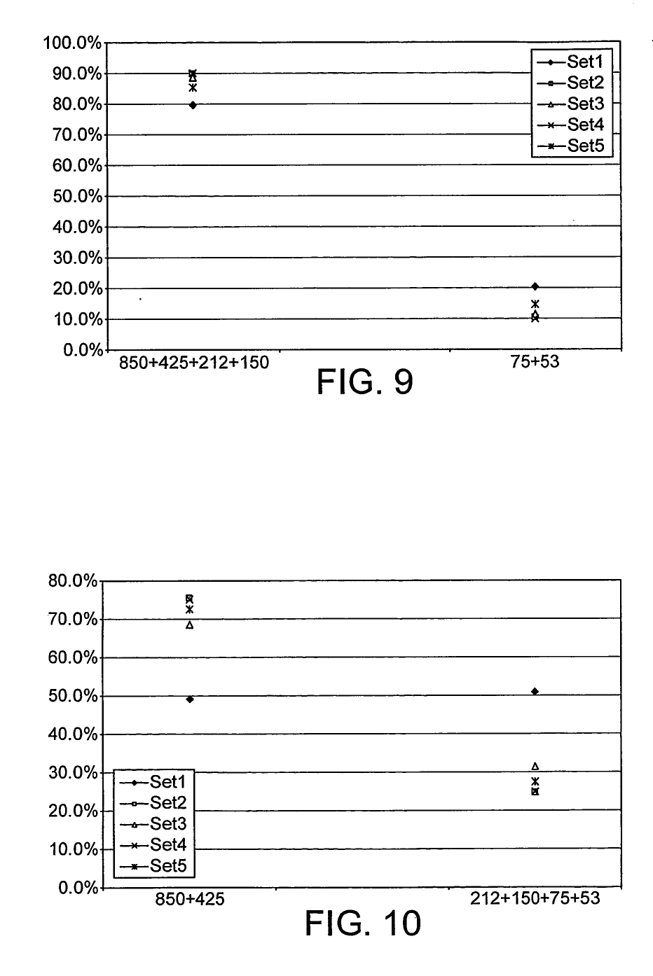

FIG. 9 compares the percentage weight distribution, when the large and intermediate particles are grouped together, versus the small particles, and

FIG. 10 compares the percentage weight distribution by separation, using a 150 μm mesh.

The experiments were performed in triplicate and a clear difference in the distribution of particles can be demonstrated.

In the table given in FIG. 4 and figures the conditions were as follows:

Set 1 Lysis 220 rpm; precipitation 440 rpm, duration 30 minutes.

Set 2 Lysis 55 rpm; precipitation 55 and 75 rpm, duration 30 minutes.

Set 3 Lysis 220 rpm; precipitation 440 rpm, duration 30 seconds.

Set 4 Lysis 100 rpm; precipitation 100 rpm, duration 30 minutes, and

Set 5 Lysis 125 rpm; precipitation 125 rpm, duration 30 minutes.

The above data demonstrates that precipitate size is affected by both the agitation rate of the impeller and the duration of the agitation.

For example: Set 1, where the agitation rate is high and the time period long, results in the formation of particles which are mainly caught on the 425 μm mesh.

This is in contrast to Set 2, where most particles are caught on the 850 μm with subsequent smaller grade meshes capturing comparatively little precipitate.

In Set 3, where the agitation rate is high but the time period short, most particles are captured on the 850 μm in the same way as for Set 2. However, there is also a greater number of particles captured on the smaller sized meshes (425, 212 and 150 μm), indicating that the effect of extended high-agitation periods is to fragment the particles with size greater than 850 μm to the 425 and 212 μm range.

Set 4 and Set 5 confirm that the low agitation rates of 100 RPM and 125 RPM can yield particles of a size generally above 850 μm.

The findings are significant, in that small particles tend to block traditional membranes.

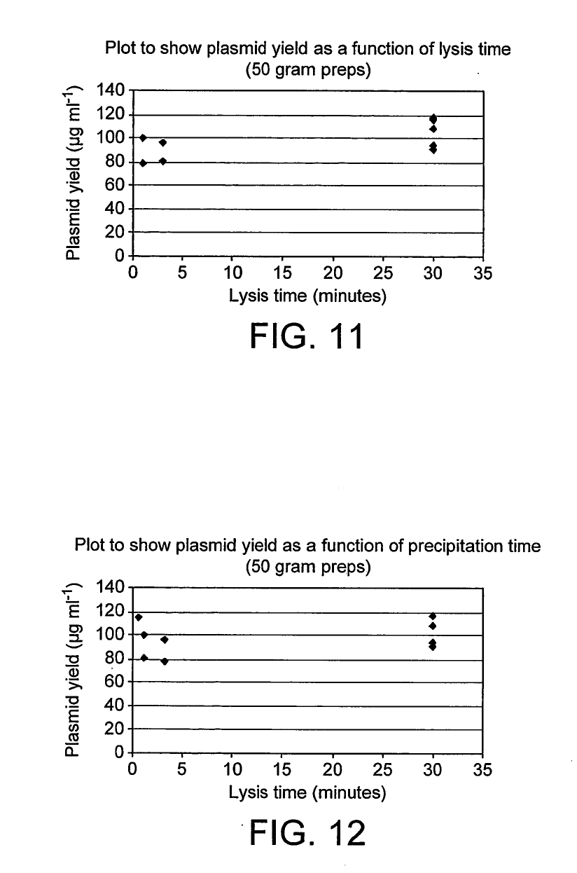

FIGS. 11 and 12 show that plasmid yield is relatively unaffected by lysis and precipitation time.

Design of Mixing Step and Subsequent Mesh Filter

It is generally easier and more economic to remove large particles from a solution than smaller ones. Therefore, in conjunction with the above data, a plate filter, agitator and operating parameters were designed in an attempt to mimic the small-scale system. The agitator and lysis vessel were designed on the basis of scaling up the 1-litre lysis vessel by maintaining dimensional homogeneity.

This was based on the following calculations:

Agitator type: Single, four bladed 60 degree pitched impeller.
Total agitator diameter (including hub):

| | | |
|---|---|---|
| Small scale equipment (mm): | agitator diameter/vessel diameter | = 58/120 |
| | | = 0.4833 |
| Agitator diameter in new mixing vessel (m): | 0.4833 × vessel diameter | = agitator diameter |
| | 0.4833 × 0.46 | = 0.2223 m |

Blade width:

| | | |
|---|---|---|
| Small scale equipment (mm): | blade width/vessel diameter | = 17/120 |
| | | = 0.1417 |
| Blade width in new mixing vessel (m): | 0.1417 × vessel diameter | = agitator diameter |
| | 0.1417 × 0.46 | = 0.0651 m |

Blade length:

| | | |
|---|---|---|
| Small scale equipment (mm): | blade length/tan—tan length | = 68/150 |
| | | = 0.4533 |
| Blade length in New mixing vessel (m): | 0.4533 × tan—tan length | = blade length |
| | 0.4533 × 0.72 | = 0.3264 m |

Ideally the vessel will be operated with identical tip speed to that in the small-scale equipment.

| | |
|---|---|
| Small-scale tip | = (rpm/60) × circumference of agitator |
| speed (m s⁻¹) | = (100/60) × 58e10⁻³ π |
| | = 0.303 m s⁻¹ |
| New mixing vessel rpm | = 0.303 × 60/0.2223 π |
| | = 26.1 rpm |

The principle outlined above can thus be used to determine an optimum impellor design for any large-scale vessel.

Since the meshes were so successful in capturing the precipitate under a gravity-fed mode to yield a process stream which was significantly reduced in solids content, a sieve system was pursued as a potential method for removal at the process scale. A scaled-up device was designed that could operate under gravity alone, or by the application of pressure.

Confirmation that the Design Works

The large-scale lab mimic of the impellor was used to confirm that precipitate of the desirable size could be made at the large scale, and that the subsequent plate filter could remove solids under a gravity-fed mode.

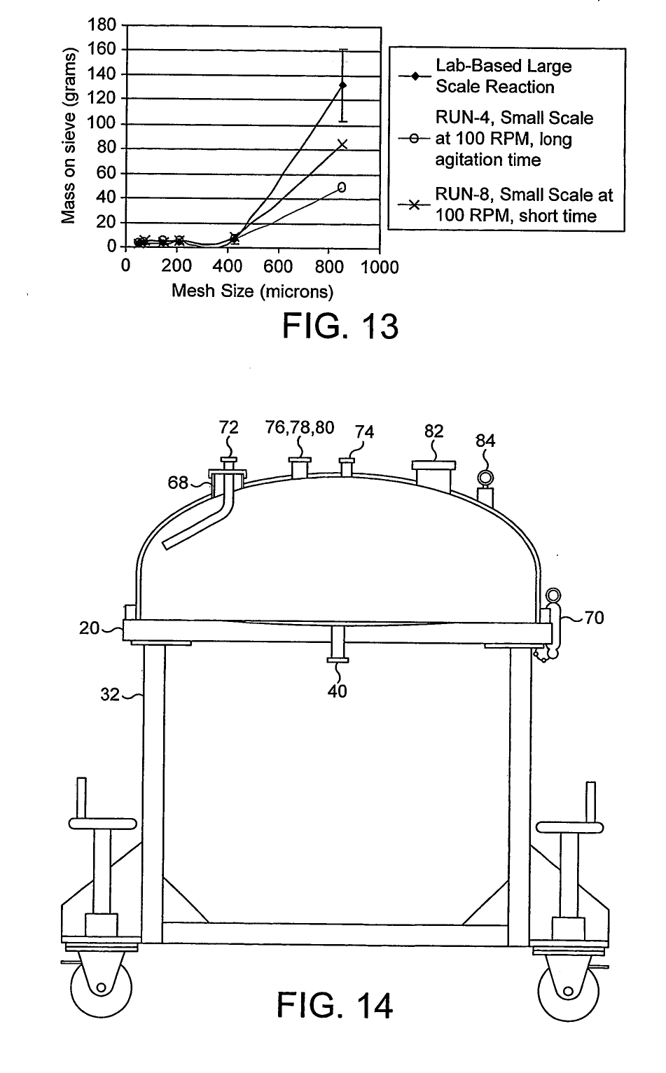

FIG. 13 shows the particle size of material originating from the large-scale vessel and compares it to that derived from the small-scale vessel. Clearly, large precipitate dominates the particle size distribution, indicating that the design of the impellor and plate filter used in the newly configured mixing step can form precipitate of the desired size.

When this material was applied to the plate filter, the precipitate was largely retained on the 200 μm mesh. A 350 mm, 200 μm mesh was able to handle 5 litres of precipitated lysate in less than 1 minute.

The sieve or mesh 10 is most clearly seen in FIG. 16. It comprises a filter mesh 12 which is spot welded to a support mesh 14 to provide it with significant rigidity. The filter mesh is a stainless steel wire mesh disc with apertures (not shown) of 200 μm. This is supported on the support mesh which is also made of stainless steel and is a 1.6 mm aperture mesh. Both are 316 grade stainless steel. A mesh seal 16 allows the sieve or mesh to be correctly aligned and seated on a filter plate 20, by sitting it in a recessed portion 46.

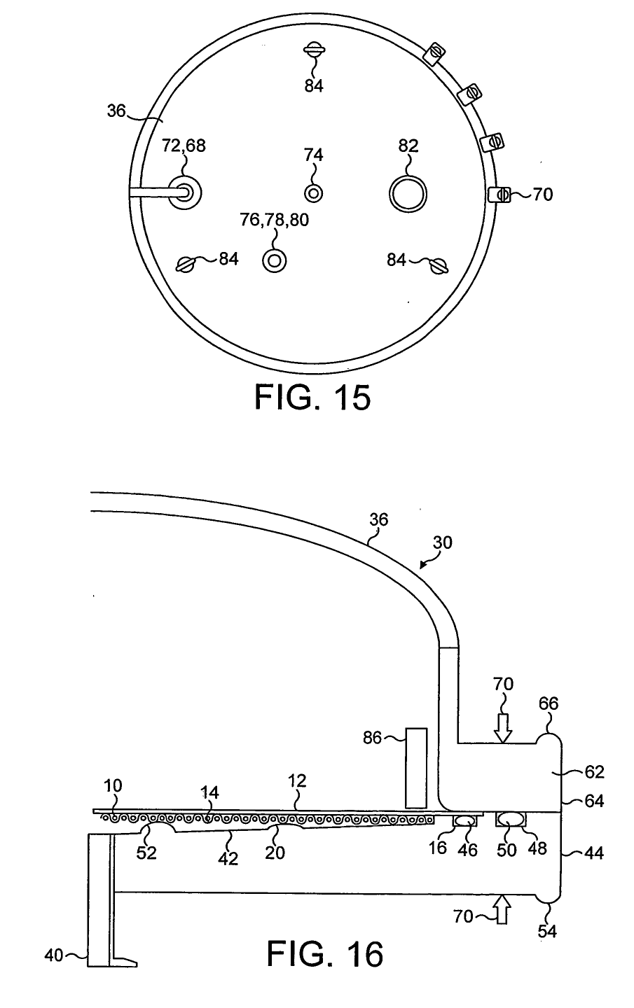

The sieve or mesh forms part of a filtration unit 30 which is illustrated in cross-section in FIG. 14, and from above in FIG. 15. The filtration unit 30 comprises a filter plate 20, a cover 36, the sieve or mesh 10 and, optionally, a stand 32.

The filter plate 20 comprises an outlet 40, a mesh abutment surface 42, which is inclined or dish-shaped so that liquid drains towards the centrally located outlet port 40. Towards its periphery 44 are two recessed portions 46 and 8. Recessed portion 46 seats mesh seal 16, and recessed portion 48 seats an O-ring 50, allowing the cover to be sealed against the filter plate 20 of the filtration unit 30. Alternatively, mesh seal 16 may take the form of a mesh seal gasket. The dished surface 42 has a gradient of about 1:50 and is provided with a plurality of spaced-apart projections 52, such as ribs, buttons or the like, which hold the mesh or sieve off the dished surface, thereby facilitating drainage.

The cover 36 comprises a flanged portion 62 at its periphery. The cover can thus be clamped against the filter plate 20 by means of a plurality of edge clamps 70. An upwardly directed lip 66 on the perimeter 64 of the cover and a downwardly directed lip 54 at the perimeter of the filter plate 20, along with the O-ring 50, help ensure a tight seal.

The cover 36 further comprises two inlets 68 and 72, a vent 74, a pressure gauge 76 and a bursting disc 78, a spare 80 and a sight glass 82. A plurality of lifting eyes 84 are also spaced about the cover.

The apparatus also comprises a filter mesh retaining ring 86.

The mesh or sieve extends across the filter plate 20, such that it is trapped between the flange 62 of the cover and the filter plate 20.

Significant features of the design include the following:
1. The provision of projections 52 on the filter plate. These limit contact area between the mesh or sieve and the filter plate, improving steam flow during steaming in plate sterilisation; and
2. The provision of a seal or gasket 16 about the edge of the mesh filter and a recessed portion 46 in the filter plate, allows accurate location and seating of the sieve. Alternatively, or in addition, a retaining ring 86 may be used to hold the sieve in place.

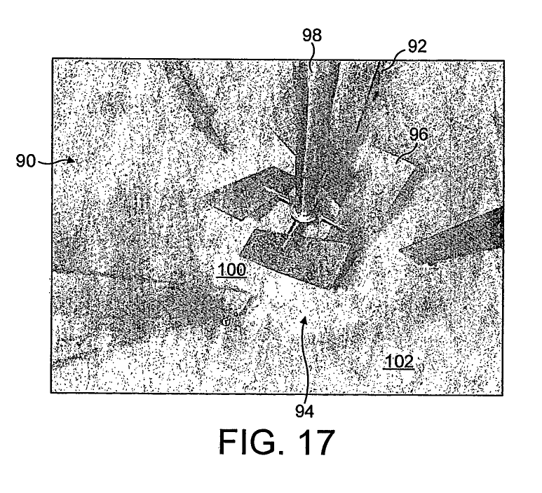

The sieve is particularly useful for use in the purification of extra chromosomal DNA e.g. pDNA.

Where the cell lysis comprises mixing the process stream with, for example, SDS, and mixing is achieved in an agitator, it is preferred that the agitator is designed to facilitate the production of ‘large’ particles. One such design is shown in FIG. 17.

Referring to FIG. 17, the agitator 90 comprises an impellor 92 which sits in a vessel 94 of 0.46 m diameter. The impellor comprises four blades 96 which are radially disposed about a shaft 98. The blades are inclined at an angle of about sixty degrees to the base 100 of the vessel. They overlap one another and are elongate in shape. Each blade is 0.326 m long and 0.065 m wide (large relative to the blades of a 1 litre vessel the blades of which are 0.068 m long and 0.017 m wide). The blades extend up the sides 102 of the vessel such that they fill at least 40% of the height of the vessel. In this manner, the process stream covers the blades during the precipitation step, but do so only partly during lysis.

The combination of a carefully designed agitator, the use of a plate filter with a mesh size of greater than 751 μm, and the control of operating conditions during the lysis and/or agitation steps to control particle size, circumvent the problems of economically feasible scale-up, speed of processing, application of mechanical stress to the precipitate and lack of integrated pipework, to successfully remove precipitated cell lysate in the manufacture of pharmaceutical plasmid DNA.

The sieve has proved particularly effective in separating the products derived from a precipitation reaction generated in the standard clarification processes employed in pDNA processing, namely high concentrations of an acidic acetate salt solution, such as, for example, sodium or potassium acetate.

It has also proved effective for separating the products of a precipitation step using an antichaotropic salt, such as, for example, ammonium sulphate, sodium sulphate, potassium citrate, calcium chloride, ammonium acetate or potassium acetate.

Indeed, in a preferred embodiment, the sieve can be used in single step to remove the solids produced by both a traditional clarification process with, for example, an acidic acetate salt solution and a RNA removal step, such as a calcium chloride precipitation. Such a step has processing advantages in a RNase free extrachromosomal DNA purification process.

1. A method of purifying extra-chromosomal DNA, by removing cell debris and/or RNA precipitate from a process stream comprising extra chromosomal DNA and cell debris precipitate and/or RNA precipitate, comprising the steps of:
Controlling a rate of agitation in the cell lysis and/or precipitation reactions, to substantially minimise the formation of small particles and/or maximise the formation of large particles wherein the small particles are those retained by a mesh size of 53 μm but which pass through a mesh size of 150 μm and the large particles are those retained by a mesh size of 425 μm and wherein the agitator comprises an impellor with large blades, pitched at an angle of from 40-80 degrees to the horizontal and extending in length to fill at least 40% of the height of a vessel in which the lysis and/or precipitation reaction is conducted and wherein the rate of agitation is maintained at a tip speed of less than 1.15 m/s; and
Straining the process stream by passing it through a mesh or sieve with a mesh size of greater than 75 μm to remove a substantial % mass of the precipitate from the process stream.

2. The method of claim 1 wherein the process stream comprises no more than 15% by weight of small particles.

3. The method of claim 1 wherein the process stream comprises at least 60% by weight of large particles.

4. The method of claim 1 wherein the process stream further comprises intermediate sized particles wherein the intermediate sized particles are those retained by a mesh size of 150 μm but which pass through a mesh or sieve with a mesh size of 425 μm.

5. The method of claim 1 wherein the process stream comprises less than 20% by weight of the intermediate particles.

6. The method of claim 1 wherein at least 50% by weight of the particles are retained by a mesh or sieve with a mesh size of 850 μm.

7. The method as claimed in claim 6 wherein at least 60% by weight of the particles are retained by a mesh or sieve with a mesh size of 850 μm.

8. The method as claimed in claim 7 wherein at least 65% by weight of the particles are retained by a mesh or sieve with a mesh size of 850 μm.

9. The method of claim 1 wherein the process stream is passed through a mesh or sieve with a mesh size of 200 μm or greater.

10. The method of claim 1 wherein the process stream is passed through a series of sieves of decreasing mesh size.

11. The method of claim 1 wherein the mesh or sieve comprises a contact face that lies substantially wholly planar to the process stream.

12. The method as claimed in claim 11 wherein the contact face is substantially rigid.

13. The method of claim 1 wherein the sieve or mesh is metal.

14. The method of claim 1 wherein the duration of agitation is controlled to less than 1 hour in the cell lysis and/or precipitation reactions to minimise the formation of small particles and maximise the formation of large particles.

15. A method of purifying extra-chromosomal DNA, by removing cell debris and/or RNA precipitate from a process stream comprising extra chromosomal DNA and cell debris precipitate and/or RNA precipitate, comprising:
a) Controlling the cell lysis and/or precipitation reactions by using static or vortex mixing, to substantially minimise the formation of small particles and/or maximise the formation of large particles wherein the small particles are those retained by a mesh size of 53 μm but which pass through a mesh size of 150 μm and the large particles are those retained by a mesh size of 425 μm; and
b) Straining the process stream by passing it through a mesh or sieve with a mesh size of greater than 75 μm to remove a substantial % mass of the precipitate from the process stream.

16. The method of claim 15 wherein the passage of the process stream to the mesh or sieve is conducted under conditions that minimise shear.

17. The method of claim 15 further comprising passing the process stream through a depth filter.

18. The method of claim 15 further comprising passing the process stream through a 0.2 μm filter membrane.

19. The method of claim 15 which omits a centrifugation step to remove cell debris.

20. The method of claim 15 in which the extra chromosomal DNA is plasmid DNA.

21. The method of claim 15 which is a large scale process comprising handling at least 10 litres of liquid in the process stream.

22. The method of claim 15 which is gravity fed.

23. The method as claimed in claim 1 which is operated under the application of pressure.

24. The method as claimed in claim 1 wherein the sieve is used to separate solids from a clarification step involving precipitation using an acidic acetate salt solution.

25. The method as claimed in claims 1 wherein the sieve is used to separate solids from a precipitation step using an antichaotropic salt to precipitate RNA.

26. The method as claimed in claim 1 wherein the sieve is used to separate solids from both:
a clarification step involving precipitation using an acidic acetate salt solution; and
a precipitation step using an antichaotropic salt, in a single precipitate removal step.

27. A method as claimed in claim 25 wherein the antichaotropic salt is calcium chloride.

28. An extra chromosomal DNA purified by a method as claimed in claim 1 or 15.

29. A pharmaceutical composition comprising or consisting of extra chromosomal DNA purified by a method as claimed in claim 1 or 15.

30. A method of purifying extrachromosomal DNA from a process stream comprising lysed cells said method comprising the steps of:
neutralising the process stream comprising lysed cells with, for example, sodium or potassium acetate;
adding a high concentration of an antichaotropic salt, for example calcium chloride, to precipitate out RNA; and
separating the solids from the process stream by passing the process stream through a mesh or sieve with a mesh size greater than 75 μm.

31. A method as claimed in claim 26 wherein the antichaotropic salt is calcium chloride.
